(19) United States
(12) Patent Application Publication
(10) Pub. No.: US 2013/0289602 A1
(43) Pub. Date: Oct. 31, 2013

(54) TREATMENT DEVICE

(71) Applicant: OLYMPUS CORPORATION, Tokyo (JP)

(72) Inventor: Masayuki KOBAYASHI, Tokyo (JP)

(21) Appl. No.: 13/924,697

(22) Filed: Jun. 24, 2013

Related U.S. Application Data
(63) Continuation of application No. PCT/JP2011/051656, filed on Jan. 27, 2011.

(30) Foreign Application Priority Data
Dec. 24, 2010 (JP) 2010-288075

(51) Int. Cl. A61B 17/34; A61B 17/28

(52) U.S. Cl. CPC A61B 17/3415; A61B 17/28
USPC 606/185

(57) ABSTRACT

Provided is a treatment device including a long, thin inserted portion having a channel that penetrates in the longitudinal direction; a forceps portion that has a pair of holding pieces that can be opened and closed at the distal end of the inserted portion; and a piercing needle that is accommodated in the channel and that is provided with a through-hole into which a guide wire can be inserted, extends in the longitudinal direction to a needle-tip face, wherein the first holding piece can be disposed at a position shifted toward the proximal end from the distal end of the second holding piece, and the piercing needle can be made to stick out from the opening at the distal-end surface of the inserted portion into a space formed between the distal ends of the pair of holding pieces, which are disposed shifted from each other.

### CROSS-REFERENCE TO RELATED APPLICATIONS

This is a continuation of International Application PCT/JP2011/051656, with an international filing date of Jan. 27, 2011, which is hereby incorporated by reference herein in its entirety. This application claims the benefit of Japanese Patent Application No. 2010-288075, the content of which is incorporated herein by reference.

### TECHNICAL FIELD

The present invention relates to a treatment device.

### BACKGROUND ART

In the related art, there is a known treatment device with which a guide wire is introduced into a pericardial cavity by piercing a pericardium in a pinched state (for example, see Patent Literature 1).

### CITATION LIST

### Patent Literature

{PTL 1} U.S. Pat. No. 5931810, Specification

### SUMMARY OF INVENTION

The present invention provides a treatment device including a long, thin inserted portion having a channel that opens at a distal-end surface thereof and that is formed in the longitudinal direction thereof; a forceps portion that is provided at the distal end of the inserted portion and that has a pair of holding pieces that can be opened and closed; and a piercing needle that is accommodated in the channel in a movable manner and that is provided with a through-hole into which a guide wire can be inserted, formed in the longitudinal direction so as to reach a needle-tip face, which is a distal-end surface thereof, wherein a first one of the holding piece is provided so that the distal end thereof can be disposed at a position shifted toward the proximal end from the distal end of a second of the holding piece, and the piercing needle is provided so that the piercing needle can be made to stick out from the opening at the distal-end surface of the inserted portion into a space formed between the distal ends of the pair of holding pieces, which are disposed so as to be relatively shifted.

### BRIEF DESCRIPTION OF DRAWINGS

FIG. 1 is an overall configuration diagram of a treatment device according to an embodiment of the present invention, showing a state in which holding pieces are open.

FIG. 2 is a cross-sectional view of the treatment device in FIG. 1 taken along I-I.

FIG. 3 shows the treatment device in FIG. 1 in a state in which the holding pieces are closed and a piercing needle is made to stick out.

FIG. 4A is a diagram for explaining a method of using the treatment device in FIG. 1, showing a state in which the treatment device has reached a pericardium.

FIG. 4B is a diagram for explaining the method of using the treatment device in FIG. 1, showing a state in which the pericardium held by the holding pieces is pierced with the piercing needle.

FIG. 4C is a diagram for explaining the method of using the treatment device in FIG. 1, showing a state in which a guide wire is introduced into a pericardial cavity.

FIG. 5A is a diagram showing a modification of a forceps portion and the piercing needle of the treatment device in FIG. 1, showing a state in which the piercing needle is accommodated in an inserted portion.

FIG. 5B shows a state in which the piercing needle is made to stick out from the inserted portion in FIG. 5A.

FIG. 6 is a diagram showing an example of a screw mechanism in the configuration of the forceps portion in FIG. 5.

FIG. 7 is a cross-sectional view of the inserted portion in FIG. 6 taken along VI-VI.

FIG. 8A is a diagram for explaining the movement of the piercing needle in FIG. 6, which shows a state in which the pericardium is held by the holding pieces.

FIG. 8B is a diagram for explaining the movement of the piercing needle in FIG. 6, which shows a state in which the pericardium is pierced with the piercing needle made to stick out from the inserted portion.

FIG. 9 is a diagram showing another modification of the forceps portion, and is a diagram showing an example configuration in which a distal-end position of a first holding piece is provided so as to be movable.

FIG. 10A is a diagram for explaining a method of using the forceps portion in FIG. 9, showing a state in which the pericardium is held by the forceps portion, with the distal end of a second holding piece disposed at a normal position.

FIG. 10B is a diagram for explaining a method of using the forceps portion in FIG. 9, showing a state in which the pericardium is pierced with the piercing needle, with the distal-end position of the second holding piece disposed at a protruding position.

### DESCRIPTION OF EMBODIMENT

A treatment device 1 according to an embodiment of the present invention will be described below with reference to the drawings.

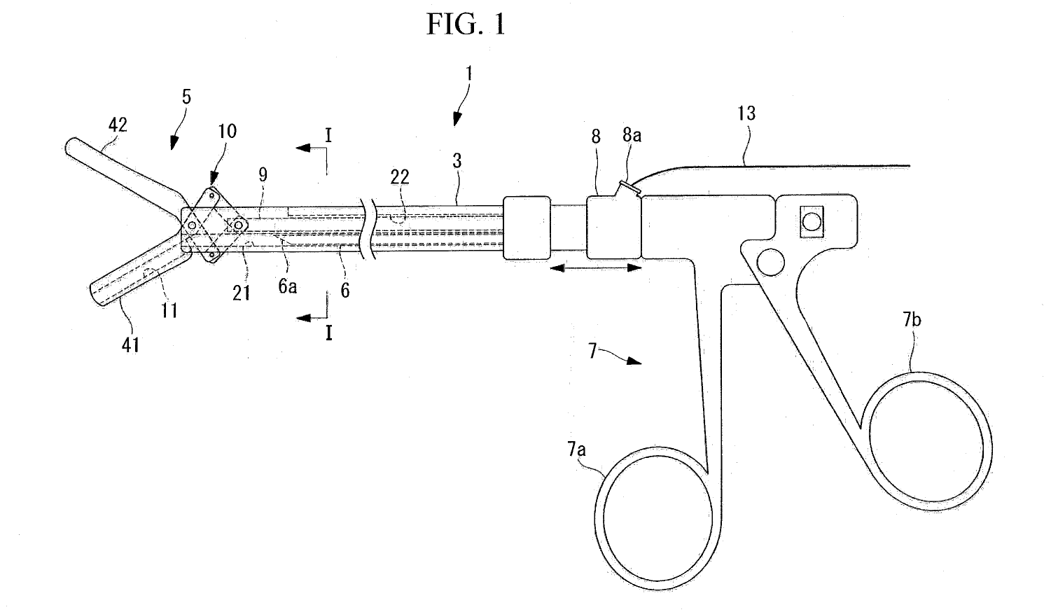

As shown in FIG. 1, the treatment device 1 according to this embodiment is provided with a long, thin inserted portion 3 having two channels 21 and 22 formed in a longitudinal direction so as to pass therethrough; a forceps portion 5 that is provided at the distal end of the inserted portion 3 and that has a pair of holding pieces 41 and 42; a piercing needle 6 that is accommodated in the inserted portion 3; and a forceps manipulating portion 7 and a needle manipulating portion 8 for manipulating the movements of the holding pieces 41 and 42 and the piercing needle 6, respectively, at the proximal end side of the inserted portion 3.

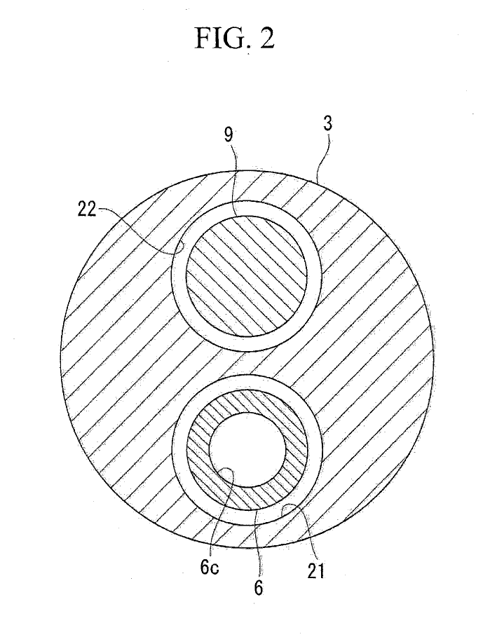

FIG. 2 is a lateral cross-sectional view of the inserted portion 3 in FIG. 1 taken through an intermediate position thereof.

The inserted portion 3 is formed of a rigid material. The first channel (channel) 21 is provided with openings at both end surfaces of the inserted portion 3, and the piercing needle 6 is inserted thereinto so as to be movable in the longitudinal direction. In the second channel 22, a holding-force transmitting member 9 that is connected to the forceps portion 5 and the forceps manipulating portion 7 at both ends thereof is inserted therethrough so as to be movable in the longitudinal direction.

The forceps portion 5 is provided with a linkage mechanism 10 that joins the pair of holding pieces 41 and 42 with each other. The linkage mechanism 10 converts a retracting movement of the holding-force transmitting member 9 toward the proximal end to a closing movement of the holding pieces 41 and 42, and converts an advancing movement of the holding-force transmitting member 9 toward the distal end to an opening movement of the holding pieces 41 and 42.

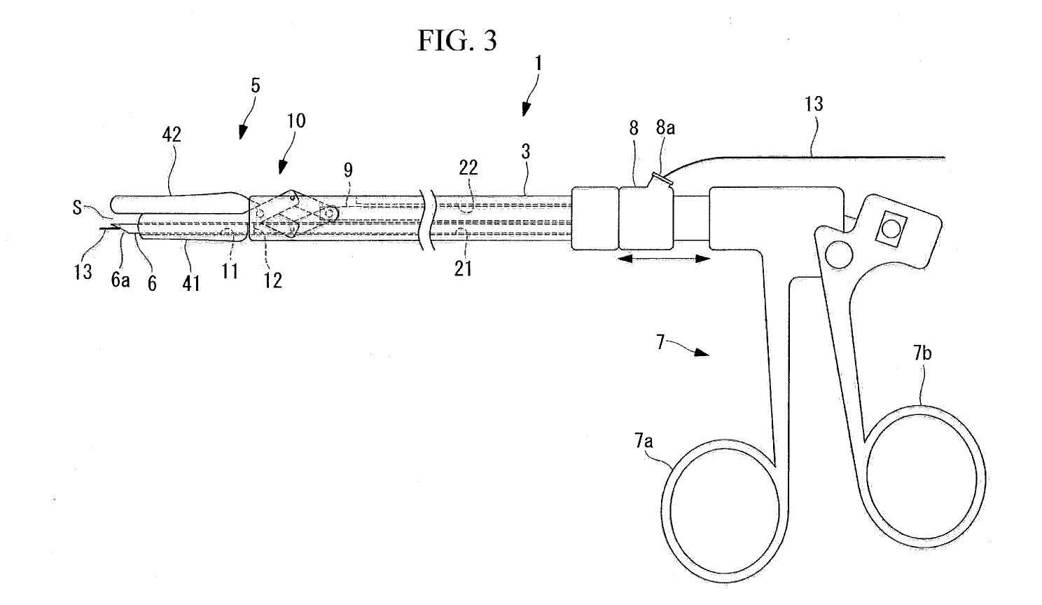

The first holding piece 41 has a smaller size than the second holding piece 42 in the longitudinal direction, and the distal end of the first holding piece 41 is disposed at a position that is shifted toward the proximal end from the distal end of the second holding piece 42. By doing so, in a state in which the holding pieces 41 and 42 are closed, as shown in FIG. 3, a space S is formed between the distal end of the first holding piece 41 and the distal end of the second holding piece 42.

In the first holding piece 41, a channel 11 is formed along the longitudinal direction thereof. In the state in which the holding pieces 41 and 42 are closed, the channel 11 is disposed at an extension of the first channel 21. The piercing needle 6 can be made to stick out from an opening at the distal end of the holding piece 41 by passing through the first channel 21 and the channel 11 of the holding piece 41. At this time, the piercing needle 6 is made to stick out into the space S.

At an outer circumferential surface of the piercing needle 6, a flange-like stopper (movement restricting member) 12 is provided, which is abutted against the edge of the opening at the distal end of the first channel 21 so as to restrict further movement of the piercing needle 6 toward the distal end when the distal end thereof is made to stick out to substantially the same position as the distal end of the second holding piece 42. By doing so, the piercing needle 6 is prevented from coming into contact with tissue in a body by protruding further than the distal end of the second holding piece 42.

As shown in FIG. 2, a through-hole 6c into which a guide wire 13 can be inserted is formed in the longitudinal direction in the piercing needle 6, and the guide wire 13 inserted into the through-hole 6c can be made to stick out from an opening at a needle-tip face 6a, that is, the distal-end surface of the piercing needle 6. The needle-tip face 6a is formed in an inclined manner with respect to the longitudinal direction, thus forming a blade face (hereinafter, also referred to as a blade face 6a).

The rotation of the piercing needle 6 in the circumferential direction is restricted by a rotation restricting mechanism so that the blade face 6a faces the first holding piece 41. The rotation restricting mechanism is configured by, for example, forming the lateral cross-sectional shapes of an inner surface of the first channel 21 and an outer surface of the piercing needle 6 in a non-circular shape, such as an elliptical shape, a polygonal shape, or the like. By doing so, because a sharp tip 6b of the piercing needle 6 made to stick out into the space S is disposed parallel to the second holding piece 42, the sharp tip 6b is prevented from coming into contact with tissue or the like disposed outside the space S.

The forceps manipulating portion 7 is provided with a fixed handle 7a and a movable handle 7b for opening and closing manipulations of the holding pieces 41 and 42. The fixed handle 7a is fixed to the inserted portion 3. The movable handle 7b is connected to the linkage mechanism 10 of the forceps portion 5 via the holding-force transmitting member 9. When an operator grips the fixed handle 7a and the movable handle 7b, the movable handle 7b is brought close to the fixed handle 7a, which causes the holding-force transmitting member 9 to be pulled toward the proximal end. Accordingly, the operator can close the holding pieces 41 and 42 by gripping the two handles 7a and 7b.

On the other hand, with the forceps manipulating portion 7, when the operator releases his/her hand from the two handles 7a and 7b, the holding-force transmitting member 9 advances, causing the holding pieces 41 and 42 to open. A fixing mechanism (not shown) that fixes the relative positions of the two handles 7a and 7b may be provided so that the holding pieces 41 and 42 are held at a certain opening angle.

The needle manipulating portion 8 is provided so as to be movable in the front-to-rear direction along the longitudinal direction of the inserted portion 3, and is also connected to the proximal end of the piercing needle 6 inside the inserted portion 3. By moving the needle manipulating portion 8 toward the proximal end, the operator can place the piercing needle 6 at an accommodated position (see FIG. 1), at which it is accommodated in the first channel 21. On the other hand, by moving the needle manipulating portion 8 toward the distal end, the operator can place the piercing needle 6 at a piercing position, at which it is made to stick out into the space S from the distal end of the first holding piece 41. In addition, the needle manipulating portion 8 has a wire introduction port 8a that communicates with the through-hole 6c of the piercing needle 6. The operator can insert the guide wire 13 into the through-hole 6c of the piercing needle 6 from the wire introduction port 8a.

Next, the operation of the thus-configured treatment device 1 will be described.

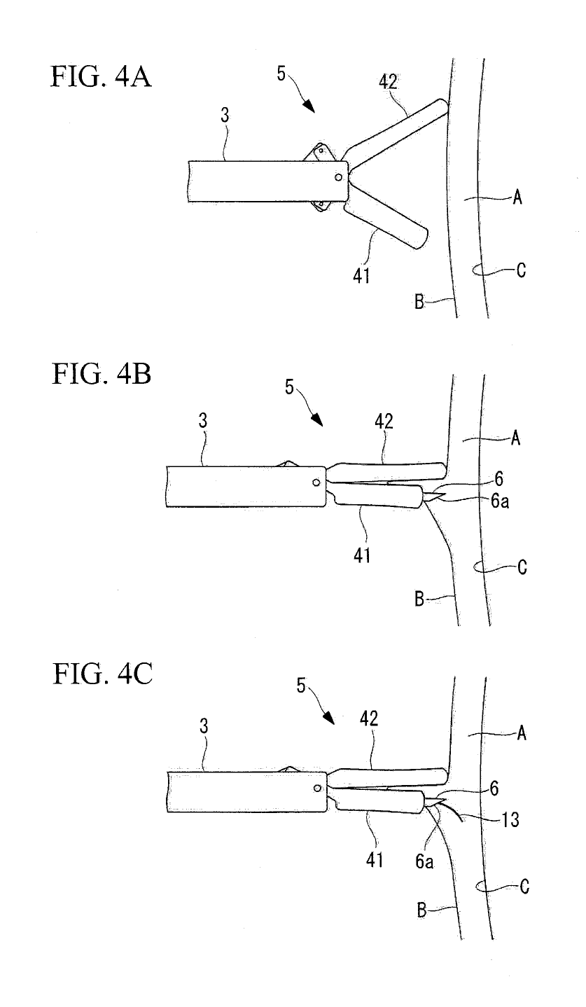

To introduce the guide wire 13 into a pericardial cavity A by using the treatment device 1 according to this embodiment, the inserted portion 3 is inserted into the body in a state in which the piercing needle 6 is disposed at the accommodated position, and the forceps portion 5 is disposed outside a pericardium B, as shown in FIG. 4A. Then, as shown in FIG. 4B, the pericardium B is held between the holding pieces 41 and 42, and the piercing needle 6 is made to stick out at the piercing position, thereby piercing the pericardium B with the piercing needle 6 so as to pass therethrough. In this state, by pushing the guide wire 13 into the wire introduction port 8a from outside the body, the guide wire 13 can be made to stick out from the distal end of the first holding piece 41 so as to be introduced into the pericardial cavity A, as shown in FIG. 4C.

In this case, with this embodiment, because the distal ends of the pair of holding pieces 41 and 42 are disposed so as to be relatively shifted in the front-to-rear direction, in the state in which the pericardium B is held therebetween, the pericardium B is pulled to a sufficiently separated position from the surface of a heart C at the distal end of the first holding piece 41, and thus, a space is formed between the pericardium B and the heart C. By doing so, the pericardium B can be pierced with the piercing needle 6, and the guide wire 13 can be introduced thereinto at the position separated from the heart C, which affords an advantage in that the distal ends of the piercing needle 6 and the guide wire 13 can be prevented from coming into contact with the heart C.

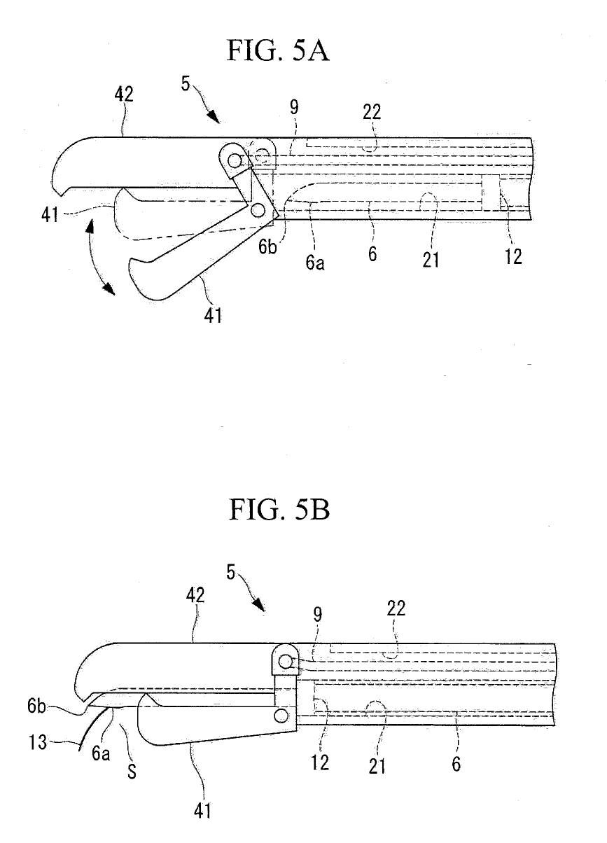

In this embodiment, the piercing needle 6 is made to stick out from the distal end of the first holding piece 41; alternatively, however, it may be made to stick out from between the pair of holding pieces 41 and 42, as shown in FIG. 5A. Also, by doing so, the piercing needle 6 can be made to stick out into the space S between the distal ends of the holding pieces 41 and 42, which are disposed so as to be relatively shifted in the front-to-rear direction, as shown in FIG. 5B, and thus, the pericardium B can be pierced with the piercing needle 6 and the guide wire 13 can be introduced into the pericardial cavity A at a position a sufficient distance away from the heart C.

In addition, in this embodiment, the blade face 6a may face sideways with respect to the longitudinal direction of the piercing needle 6 by forming the distal-end portion of the piercing needle 6 so as to be curved in one direction, as shown in FIGS. 5A and 5B. In this case, the rotation of the piercing needle 6 is restricted by the rotation restricting mechanism so that the blade face 6a faces the first holding piece 41.

By doing so, the guide wire 13 sticks out sideways with respect to the longitudinal direction of the inserted portion 3, as shown in FIG. 5B. In other words, because the guide wire 13 is introduced into the pericardial cavity A along a direction away from the heart C, which is disposed forward of the holding pieces 41 and 42, the distal end of the guide wire 13 can more reliably be prevented from coming into contact with the heart C.

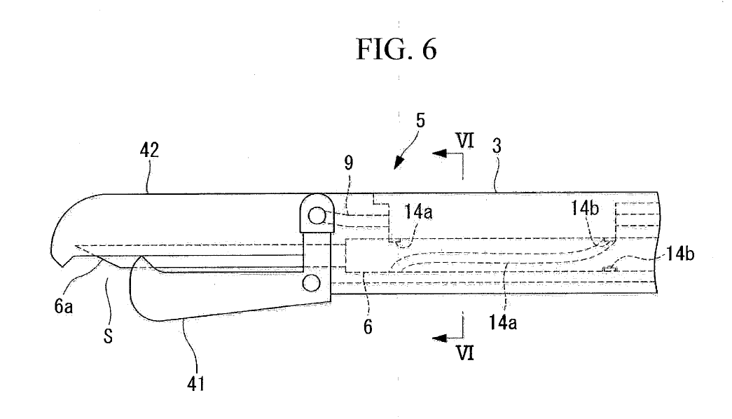

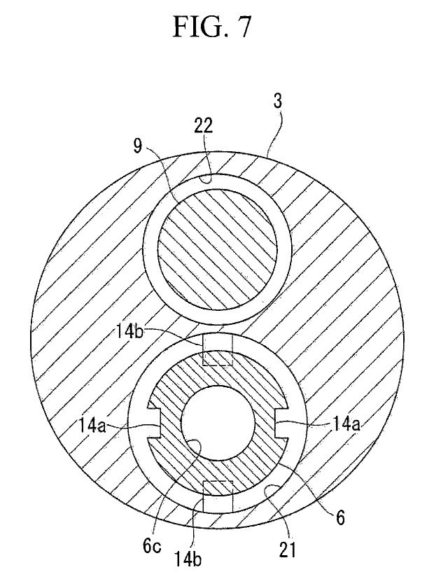

In addition, in this embodiment, the rotation of the piercing needle 6 is restricted by the rotation restricting mechanism so that the blade face 6a faces the first holding piece 41; alternatively, however, a screw mechanism that moves the piercing needle 6 forward and backward while rotating it in the circumferential direction may be provided. As shown in FIG. 6, the screw mechanism is constituted of, for example, a male thread 14a including spiral grooves formed on a side surface of the piercing needle 6 and a female thread 14b that is provided at the inner surface of the first channel 21 and that is engaged with the male thread 14a. FIG. 7 is a lateral cross-sectional view of the inserted portion 3 taken through an intermediate position thereof. The screw mechanism is configured so that the blade face 6a faces the second holding piece 42 at the accommodated position and the blade face 6a faces the first holding piece 41 at the piercing position.

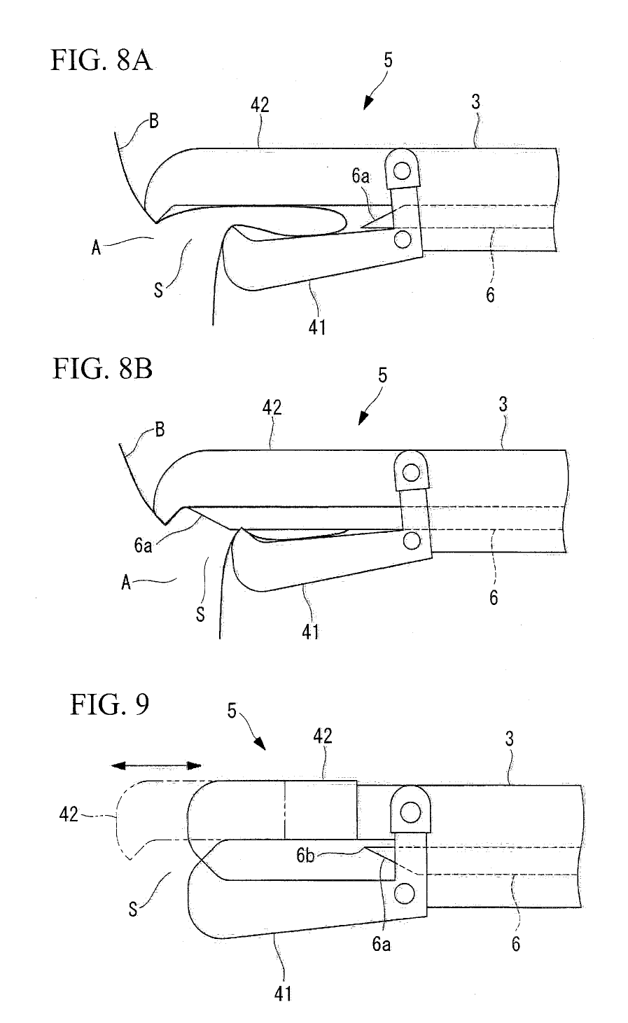

By doing so, as shown in FIG. 8A, the pericardium B is pierced with the piercing needle 6 at a position where it is pulled to a position sufficiently far away from the surface of the heart C by means of the first holding piece 41. As shown in FIG. 8B, the blade face 6a is subsequently made to face the first holding piece 41 by being advanced to the piercing position while being rotated. In other words, by piercing the flexible pericardium B at a position at which it has a greater tension, the piercing needle 6 can be made to more reliably pass through the pericardium B.

In addition, in this embodiment, the second holding piece 42 may be provided so as to be movable in the front-to-rear direction at the distal-end position thereof, as shown in FIG. 9. The second holding piece 42 is provided so as to be movable between a normal position and a protruding position. At the normal position, the distal end of the second holding piece 42 is disposed in alignment with the distal end of the first holding piece 41. At the protruding position, the distal end of the second holding piece 42 is disposed so as to protrude further than the distal end of the first holding piece 41. The second holding piece 42 may be movable continuously between the normal position and the protruding position. Alternatively, the second holding piece 42 maybe movable stepwise from the normal position to a plurality of protruding positions with different protrusion levels by means of a click mechanism (not shown) or the like.

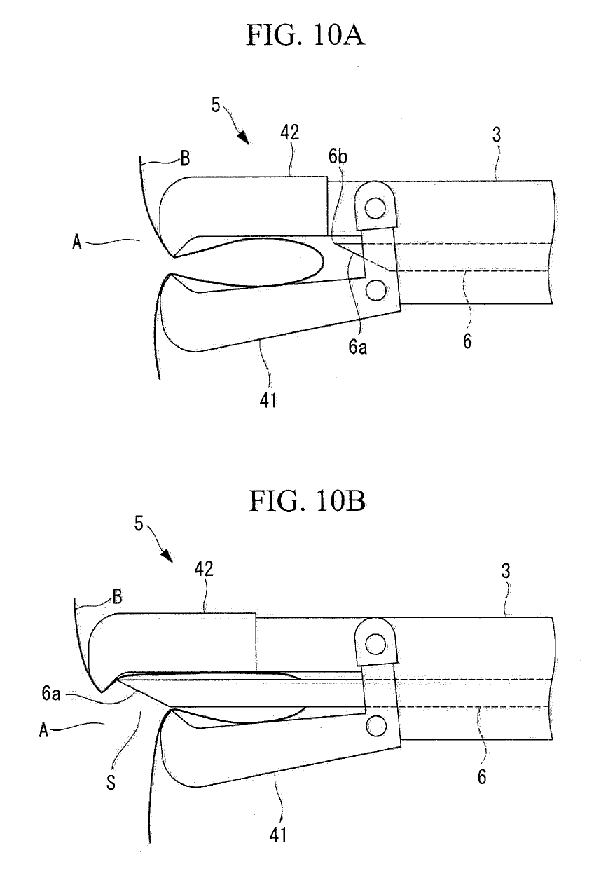

With the thus-configured forceps portion 5, as shown in FIG. 10A, it is inserted until reaching the pericardium B and the pericardium B is held in a state in which the second holding piece 42 is disposed at the normal position. As shown in FIG. 10B, by subsequently moving the second holding piece 42 to the protruding position in the state in which the pericardium B is held, the pericardium B can be pierced with the piercing needle 6 at a position sufficiently far away from the heart C.

In this way, by selecting the distal-end positions of the holding piece 42 depending on the specifics of individual manipulations, it is possible to more easily perform manipulations, such as holding of the pericardium B or the like.

In this case, it is preferable that the movement restricting member that restricts the protrusion levels of the piercing needle 6 to be made to stick out into the space S also be moved in association with the movement of the distal-end position of the second holding piece 42. For example, an abutting member (not shown) against which the stopper 12 provided at the outer circumferential surface of the piercing needle 6 is abutted may be provided so as to be movable inside the first channel 21 together with the second holding piece 42. By doing so, when the distal-end position of the second holding piece 42 is moved, the sharp tip 6b of the piercing needle 6 can be made to stick out to a certain position with respect to the distal-end position.

The following aspects of invention are derived from the embodiment and modifications thereof described above.

The present invention provides a treatment device including a long, thin inserted portion having a channel that opens at a distal-end surface thereof and that is formed in the longitudinal direction thereof; a forceps portion that is provided at the distal end of the inserted portion and that has a pair of holding pieces that can be opened and closed; and a piercing needle that is accommodated in the channel in a movable manner and that is provided with a through-hole into which a guide wire can be inserted, extends in the longitudinal direction to a distal-end surface thereof serving as a needle-tip face, wherein a first one of the holding piece is provided so that the distal end thereof can be disposed at a position shifted toward the proximal end from the distal end of a second of the holding piece, and the piercing needle is provided so that the piercing needle can be made to stick out from the opening at the distal-end surface of the inserted portion into a space formed between the distal ends of the pair of holding pieces, which are disposed so as to be relatively shifted.

With the present invention, by inserting the inserted portion into a body and by making the piercing needle stick out from the channel so as to pass through a pericardium in a state in which the pericardium is held between the pair of holding pieces of the forceps portion, the guide wire can be introduced into a pericardial cavity via the through-hole in the piercing needle.

In this case, at the first holding piece holding the pericardium, a space is formed between the pericardium and a heart by an amount corresponding to the amount by which the distal end of the first holding piece is withdrawn from the distal end of the second holding piece, and the guide wire is made to stick out into this space from the needle-tip face of the piercing needle. By doing so, the guide wire can be introduced into the pericardial cavity while preventing the distal end of the guide wire from coming into contact with the heart.

The above-described invention may be provided with a movement restricting member that restricts movement of the piercing needle toward the distal end further than the distal end of the second holding piece.

By doing so, the piercing needle can be prevented from protruding toward the distal end further than the distal end of the second holding piece.

In the above-described configuration, the piercing needle may be provided with a blade face constituted of the needle-tip face formed in an inclined manner with respect to the longitudinal direction and the treatment device may be provided with a rotation restricting mechanism that restricts rotation of the piercing needle in the circumferential direction so that the blade face of the piercing needle, when made to stick out into the space, faces the first holding piece.

By doing so, because a sharp tip of the blade face of the piercing needle is disposed closer to the second holding piece at the position at which it made to stick out from the channel, the sharp tip of the piercing needle can be prevented from coming into contact with tissue.

In the above-described configuration, in which the rotation restricting mechanism is provided, the rotation restricting mechanism may be provided with a screw mechanism that moves the piercing needle forward and backward while rotating the piercing needle in the circumferential direction so that the blade face faces the second holding piece at a position where the piercing needle is accommodated in the channel, and so that the blade face faces the first holding piece at a position where the piercing needle is made to stick out into the space.

By doing so, the pericardium is pierced with the sharp tip of the piercing needle at a position where the pericardium is pulled to a greater extent. Accordingly, the pericardium can be more reliably pierced with the piercing needle.

In addition, in the above-described invention, the piercing needle may be curved at the distal-end portion so that the needle-tip face faces sideways and may be provided with a rotation restricting mechanism that restricts rotation of the piercing needle in the circumferential direction so that the needle-tip face of the piercing needle faces the first holding piece.

By doing so, in the space between the heart and the pericardium formed at the first holding piece, because the guide wire is introduced in a direction away from the heart, the distal end of the guide wire can more reliably be prevented from coming into contact with the heart.

In addition, in the above-described invention, the second holding piece may be provided so as to be movable between a normal position, at which the distal end thereof is disposed in alignment with the distal end of the first holding piece, and a protruding position, at which the distal end thereof is disposed protruding so as to protrude toward the distal end further than distal end of the first holding piece.

By doing so, the ease of manipulation can be enhanced by selecting the distal-end position of the second holding piece depending on the specifics of manipulations, for example, by aligning the distal ends of the holding pieces when holding the pericardium, by moving the second holding piece so as to shift the distal-end position thereof when piercing the pericardium with the piercing needle, and so-on.

In the above-described configuration, the second holding piece may be provided so as to be movable to a plurality of the protruding positions at which the distal end thereof and the distal end of the first holding piece are shifted by different amounts.

By doing so, the ease of manipulation can be enhanced by selecting the amount by which the distal ends of the pair of holding pieces are shifted depending on the specifics of manipulations.

In the above-described configuration, in which the second holding piece is provided so as to be movable to the plurality of protruding positions, the treatment device may be provided with a movement restricting member that restricts movement of the piercing needle toward the distal end further than the distal end of the second holding piece, and the movement restricting member may be provided so as to be movable together with the second holding piece.

By doing so, protrusion levels of the piercing needle from the distal end of the second holding piece can be restricted within a certain range irrespective of the distal-end position of the second holding piece.

### REFERENCE SIGNS LIST

1 treatment device

3 inserted portion

5 forceps portion

6 piercing needle

6a needle-tip face, blade face

6b sharp tip

6c through-hole

7 forceps manipulating portion

7a fixed handle

7b movable handle

8 needle manipulating portion

8a wire introduction port

9 holding-force transmitting member

10 linkage mechanism

11 channel

12 stopper (movement restricting member)

13 guide wire

14a male thread (screw mechanism, rotation restricting mechanism)

14b female thread (screw mechanism, rotation restricting mechanism)

21, 22 channel

41, 42 holding piece

A pericardial cavity

B pericardium

C heart

S space

1. A treatment device comprising:
a long, thin inserted portion having a channel that opens at a distal-end surface thereof and that is formed in the longitudinal direction thereof;
a forceps portion that is provided at the distal end of the inserted portion and that has a pair of holding pieces that can be opened and closed; and
a piercing needle that is accommodated in the channel in a movable manner and that is provided with a through-hole into which a guide wire can be inserted, extends in the longitudinal direction to a distal-end surface thereof serving as a needle-tip face,
wherein a first one of the holding piece is provided so that the distal end thereof can be disposed at a position shifted toward the proximal end from the distal end of a second of the holding piece, and
the piercing needle is provided so that the piercing needle can be made to stick out from the opening at the distal-end surface of the inserted portion into a space formed between the distal ends of the pair of holding pieces, which are disposed so as to be relatively shifted.

2. The treatment device according to claim 1, further comprising a movement restricting member that restricts movement of the piercing needle toward the distal end further than the distal end of the second holding piece.

3. The treatment device according to claim 2,
wherein the piercing needle is provided with a blade face constituted of the needle-tip face formed in an inclined manner with respect to the longitudinal direction and
is provided with a rotation restricting mechanism that restricts rotation of the piercing needle in the circumferential direction so that the blade face of the piercing needle, when made to stick out into the space, faces the first holding piece.

4. The treatment device according to claim 3, wherein the rotation restricting mechanism is provided with a screw mechanism that moves the piercing needle forward and backward while rotating the piercing needle in the circumferential direction so that the blade face faces the second holding piece at a position where the piercing needle is accommodated in the channel, and so that the blade face faces the first holding piece at a position where the piercing needle is made to stick out into the space.

5. The treatment device according to claim 1,
wherein the piercing needle is curved at the distal-end portion so that the needle-tip face faces sideways and
is provided with a rotation restricting mechanism that restricts rotation of the piercing needle in the circumferential direction so that the needle-tip face of the piercing needle faces the first holding piece.

6. The treatment device according to claim 1, wherein the second holding piece is provided so as to be movable between a normal position, at which the distal end thereof is disposed in alignment with the distal end of the first holding piece, and a protruding position, at which the distal end thereof is disposed protruding so as to protrude toward the distal end further than distal end of the first holding piece.

7. The treatment device according to claim 6, wherein the second holding piece is provided so as to be movable to a plurality of the protruding positions at which the distal end thereof and the distal end of the first holding piece are shifted by different amounts.

8. The treatment device according to claim 7, further comprising a movement restricting member that restricts movement of the piercing needle toward the distal end further than the distal end of the second holding piece,
wherein the movement restricting member is provided so as to be movable together with the second holding piece.
